(19) United States
(12) Patent Application Publication
(10) Pub. No.: US 2002/0046412 A1
(43) Pub. Date: Apr. 18, 2002

(54) Nucleic acids encoding new insect acetylcholine receptor beta subunits

(76) Inventors: Martin Adamczewski, Koln (DE); Christoph Methfessel, Wuppertal (DE); Thomas Schulte, Koln (DE)

Correspondence Address: BAYER CORPORATION, PATENT DEPARTMENT, 100 BAYER ROAD, PITTSBURGH, PA, 15205, US

(21) Appl. No.: 09/732,680

(22) Filed: Dec. 8, 2000

(30) Foreign Application Priority Data
Dec. 10, 1999 (DE) 19959582.8

(51) Int. Cl. C07H 21/04; A01K 67/033; C12P 21/02; C07K 14/705; C12N 5/06

(52) U.S. Cl. 800/13; 435/69.1; 435/325; 530/350; 536/23.5

(57) ABSTRACT

The invention relates to nucleic acids which encode insect acetylcholine receptor β subunits, and to polypeptides which exert the biological function of such acetylcholine receptor β subunits, and in particular to their use for finding active compounds for crop protection.

The invention relates to nucleic acids encoding insect acetylcholine receptor β subunits and to polypeptides which have the biological function of such acetyl-choline receptor β subunits, and in particular to their use for finding active compounds for crop protection.

Nicotinergic acetylcholine receptors are ligand-controlled ion channels which play a role in neurotransmission in the animal kingdom. The binding of acetylcholine or other agonists to the receptor causes temporary opening of the channel and allows cations to pass through. It is assumed that a receptor is composed of five subunits arranged around a pore. Each of these subunits is a protein which is composed of an extracellular N-terminal moiety, followed by three transmembrane regions, an intracellular moiety, and a fourth transmembrane region and a short extracellular C-terminal moiety. Certain subunits carry the binding site for ligands, such as acetylcholine, on their extracellular moiety. Two vicinal cysteines are a component of this binding site, and therefore a joint structural feature for all ligand-binding subunits, which are also termed a-subunits. Depending on localization and function of the receptor, subunits without this structural feature are termed β, γ, δ or ε subunits (Changeux et al. 1992).

Acetylcholine receptors have been the subject of many studies, in particular in vertebrates. Owing to their anatomical localization and their functional properties (conductive properties of the channel, desensitization, sensitivity to agonists and antagonists, and to toxins such as, for example, α-bungarotoxin), three groups can be distinguished. The classification correlates with the molecular composition of the receptors. There are heterooligomeric receptors with the subunit composition α₂βγδ, which are found in muscle (Noda et al. 1982, Claudio et al. 1983, Devillers-Thiery et al. 1983, Noda et al. 1983a, b), heterooligomeric receptors which contain subunits from group α2-α6 and β2-β4 and which are found in the nervous system CD (Schoepfer et al. 1990, Heinemann et al. 1997), and homooligomeric receptors which contain subunits from group α7-α9 and which are also found in the nervous system (Lindstrom et al. 1997, Elgoyhen et al. 1997). This classification is also supported when taking into consideration the relationship of the gene sequences of the various subunits. Typically, the sequences of functionally homologous subunits of different species show greater similarity than sequences of subunits from different groups, but of the same species. Furthermore, the gene sequences of all known acetylcholine receptor subunits do not just resemble each other somewhat, but also resemble those of some other, ligand-controlled ion channels (for example the serotonin receptors of the 5HT₃ type, the GABA-controlled chloride channels, the glycine-controlled chloride channels). It can therefore be assumed that all these receptors originate from a joint precursor, and they are classified in a supergene family (Ortells et al. 1995).

In insects, acetylcholine is the most important excitatory neurotransmitter of the central nervous system. Accordingly, acetylcholine receptors can be detected electrophysiologically in preparations of insect central ganglia. This is detected successfully both on post- and on presynaptic nerve endings and on the cytosomes of intemeurons, motoneurons and modulatory neurons. (Breer et al. 1987, Buckingham et al. 1997). The receptors include those which are inhibited by α-bungarotoxin and those which are insensitive (Schloβ et al. 1988). Moreover, the acetylcholine receptors are the molecular target for important natural (for example nicotine) and synthetic insecticides (for example chloronicotinyls).

The gene sequences of a number of insect nicotinic acetylcholine receptors are already known. Thus, the sequences of five different subunits are described for Drosophila melanogaster (Bossy et al. 1988, Hermanns-Borgmeyer et al. 1986, Sawruk et al. 1990a, 1990b, Schulz et al. 1998); five sequences are also described for Locusta migratoria (Hermsen et al. 1998), one for Schistocerca gregaria (Marshall et al. 1990), six for Myzus persicae (Sgard et al. 1998, Huang et al. 1999), two for Manduca sexta (Eastham et al. 1997, Genbank AJ007397) and six for Heliothis virescens (DE 198 19 829, Genbank AF143846, AF143847, AJ000399, AF096878, AF096879). Moreover, a series of Drosophila melanogaster partial gene sequences has been characterized as expressed sequence tags (Genbank AA540687, AA698155, AA697710, AA697326). These sequences can be classified into α and β subunits, depending on whether the two vicinal cysteines of the ligand binding site are present or not. However, while a total of four different β-subunits are known in vertebrates (Schoepfer et al. 1990, Heinemann et al. 1997), at least five in Caenorhabditis elegans (Bargmann and Kaplan 1998), only one β-subunit in each case has been identified in each of the insect species investigated, with the exception of Drosophila melanogaster (Huang et al., 1999, Hermsen et al. 1998, Genbank AJ007397). This common subunit is a homologue of the subunit termed ARD in the case of Drosophila melanogaster (Schloβ et al. 1988). The sequence of the other Drosophila β subunit, SBD (Sawruk et el. 1990b), shows greater similarity with the a subunits than with the other β subunit, but does not have the vicinal cysteines.

The recombinant expression of insect nicotinic receptors has proved to be more difficult than that of the analogous vertebrate or C. elegans receptors. Thus, it has generally not been possible to express nicotinic receptors only composed of insect subunits in such a manner that their functional properties such as, for example, sensitivity show similarity with those of natural receptors (Amar et al. 1995, Hermsen et al. 1998, Sgard et al. 1998). However, at least some α subunits from various insect species contribute to a functional receptor when a chicken β2 subunit rather than an insect β subunit is coexpressed in Xenopus oocytes (Marshall et al. 1990, Schulz et al. 1998, Matsuda et al. 1998). This as well as the fact that essentially only one insect β subunit is known, allows the speculation that further, as yet unknown, β subunits exist.

The functional expression of insect nicotinic receptors in eukaryotic cell lines or Xenopus laevis oocytes is of great practical importance, for example in the search for new insecticides.

The present invention is therefore based in particular on the object of providing nucleic acids which encode novel insect acetylcholine receptor β subunits. These new nucleic acids are intended to allow the recombinant expression of nicotinic acetylcholine receptors which are composed exclusively of insect subunits.

The object is achieved by providing a nucleic acid comprising a sequence selected from the group consisting of

(a) the sequence of SEQ ID NO: 1,

(b) subsequences of the sequence defined under (a) which are at least 14 base-pairs in length,

(c) sequences which hybridize with the sequence defined under (a),

(d) sequences which have at least 70% identity to the sequence between position 43 and position 1368 of the sequence defined under (a),

(e) sequences which are complementary to the sequence defined under (a), and

(f) sequences which, owing to the degeneracy of the genetic code, encode the same amino acid sequence as do the sequences defined under (a) to (d).

The nucleic acids according to the invention are, in particular, single- or double-stranded deoxyribonucleic acids (DNA) or ribonucleic acids (RNA). Preferred embodiments are fragments of genomic DNA which can contain introns, and cDNAs.

A preferred embodiment of the nucleic acids according to the invention is the cDNA which has the nucleic acid sequence of SEQ ID NO: 1.

The degree of identity of the nucleic acid sequences is preferably determined with the aid of the program GAP from the package GCG, version 10.0, using standard settings (Devereux et al. 1984).

The term “to hybridize” as used in the present context describes the process during which a single-stranded nucleic acid molecule undergoes basepairing with a complementary strand. Starting from the sequence information disclosed herein, this allows, for example, DNA fragments to be isolated from insects other than Drosophila melanogaster which encode polypeptides with the biological function of acetylcholine receptor β subunits.

Preferred hybridization conditions are stated hereinbelow:

Hybridization solution: 6X SSC/0% formamide, preferred hybridization solution: 6X SSC/25% formamide

Hybridization temperature: 34° C., preferred hybridization temperature: 42° C.

Wash step 1: 2X SSC at 40° C.,

Wash step 2: 2X SSC at 45° C.; preferred wash step 2: 0.6X SSC at 55° C.; especially preferred wash step 2: 0.3X SSC at 65° C.

The present invention encompasses nucleic acids which have at least 70% identity, preferably at least 80% identity, especially preferably at least 90% identity, very especially preferably at least 95% identity, to the sequence between position 43 and position 1368 of the sequence of SEQ ID NO: 1, preferably over a length of at least 100, especially preferably at least 500, consecutive nucleotides, and very especially preferably over the entire length.

Subject-matter of the invention are furthermore vectors which contain at least one of the nucleic acids according to the invention. Vectors which can be used are all plasmids, phasmids, cosmids, YACs or artificial chromosomes used in molecular biology laboratories. To express the nucleic acid according to the invention, the latter can be linked to customary regulatory sequences. The choice of such regulatory sequences depends on whether pro- or eukaryotic cells or cell-free systems are used for expression. Especially preferred as expression control sequence are, for example, the SV40, adenovirus or cytomegalovirus early or late promoter, the lac system, the trp system, the main operator and promoter regions of the phage lambda, the fd coat protein control regions, the 3-phosphoglycerate kinase promoter, the promoter of acid phosphotase and the promoter of the yeast α-mating factor, the Baculovirus immediate early promoter, and the Drosophila melanogaster metallothionine promoter.

To express the nucleic acid according to the invention, the latter can be introduced into suitable host cells. Suitable host cells are not only prokaryotic cells, preferably E. coli, but also eukaryotic cells, preferably mammalian or insect cells. Other examples of suitable single-celled host cells are: Pseudomonas, Bacillus, Streptomyces, yeasts, HEK-293, Schneider S2, SF9, CHO, COS1 and COS7 cells, plant cells in cell culture, and amphibian cells, in particular oocytes.

Subject-matter of the present invention are also the polypeptides encoded by the nucleic acid according to the invention.

Subject-matter of the present invention are also polypeptides which encompass an amino acid sequence and have at least 40% identity, preferably at least 60% identity, especially preferably at least 80% identity, to the sequence of SEQ ID NO: 2 over a length of at least 20, preferably at least 25, especially preferably at least 30, consecutive amino acids, and very especially preferably over the entire length.

The degree of identity of the amino acid sequences is preferably determined with the aid of the program GAP from the package GCG, version 10.0, using standard settings (Devereux et al. 1984).

Subject-matter of the present invention are furthermore acetylcholine receptors which encompass the polypeptides according to the invention.

The term “polypeptides” as used in the present context not only relates to short amino acid chains which are usually termed peptides, oligopeptides or oligomers, but also to longer amino acid chains which are usually termed proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP-ribosylations, amidations, covalent linkages to flavins, haem moieties, nucleotides or nucleotide derivatives, lipids or lipid derivatives or phosphatidylinositol, cyclizations, di-sulphide bridge formations, demethylations, cystin formations, formylations, gamma-carboxylations, glycosylations, hydroxylations, iodinations, methylations, my-ristylations, oxidations, proteolytic processings, phosphorylations, selenylations and tRNA-mediated additions of amino acids.

The polypeptides according to the invention may exist in the form of “mature” proteins or as parts of larger proteins, for example as fusion proteins. They can furthermore exhibit secretion or leader sequences, pro-sequences, sequences which allow simple purification, such as multiple histidine residues, or additional stabilizing amino acids.

The polypeptides according to the invention need not constitute complete acetyl-choline receptor β-subunits, but may also be mere fragments thereof, as long as they can at least still exert the biological function of the complete subunits.

The polypeptides according to the invention need not be obtainable from Drosophila melanogaster acetylcholine receptor β subunits. Polypeptides which correspond to acetylcholine receptor β subunits of other insects, or fragments of these which can still exert the biological function of these subunits, are also considered to be in accordance with the invention.

In comparison with the corresponding region of naturally occurring acetylcholine receptor β subunits, the polypeptides according to the invention may exhibit deletions or amino acid substitutions, as long as they at least still exert the biological function of the complete subunits. Conservative substitutions are preferred. Such conservative substitutions encompass variations, one amino acid being replaced by another amino acid from amongst the following group:

1. Small aliphatic residues, nonpolar residues or residues of little polarity: ala, ser, thr, pro and gly;

2. Polar, negatively charged residues and their amides: asp, asn, glu and gln;

3. Polar, positively charged residues: his, arg and lys;

4. Large aliphatic nonpolar residues: met, leu, ile, val and cys; and

5. Aromatic residues: phe, tyr and trp.

Preferred conservative substitutions can be seen from the following list:

| Original residue | Substitution |
|---|---|
| ala | gly, ser |
| arg | lys |
| asn | gln, his |
| asp | glu |
| cys | ser |
| gln | asn |
| glu | asp |
| gly | ala, pro |
| his | asn, gln |
| ile | leu, val |
| leu | ile, val |
| lys | arg, gln, glu |
| met | leu, tyr, ile |
| phe | met, leu, tyr |
| ser | thr |
| thr | ser |
| trp | tyr |
| tyr | trp, phe |
| val | ile, leu |

The term “biological function of an acetylcholine receptor β subunit” as used in the present context means a role in generating functional acetylcholine receptors, that is, the ability of being able to interact with other subunits of the receptors.

A preferred embodiment of the polypeptides according to the invention is a Drosophila melanogaster acetylcholine receptor β subunit which has the amino acid sequence of SEQ ID NO: 2.

Subject-matter of the present invention are furthermore processes for producing the polypeptides according to the invention. To produce the polypeptides encoded by the nucleic acid according to the invention, host cells which contain the nucleic acid according to the invention can be cultured under suitable conditions. Thereupon, the desired polypeptide can be isolated in the customary manner from the cells or the culture medium.

A rapid method of isolating the polypeptides according to the invention, which are synthesized by host cells using a nucleic acid according to the invention, starts with the expression of a fusion protein, it being possible for the fusion component to be affinity-purified in a simple manner. For example, the fusion component may be glutathione S-transferase. The fusion protein can then be purified on a glutathione affinity column. The fusion component can be removed by partial proteolytic cleavage, for example on linkers between the fusion component and the polypeptide according to the invention to be purified. The linker can be designed such that it includes target amino acids such as arginine and lysine residues, which define sites for trypsin cleavage. To generate such linkers, standard cloning methods using oligo-nucleotides may be employed.

Other purification methods which are possible are based on preparative electrophoresis, FPLC, HPLC (for example using gel filtration, reversed-phase or moderately hydro-phobic columns), gel filtration, differential precipitation, ion-exchange chromatography and affinity chromatography.

The purification of the polypeptides according to the invention can encompass the isolation of membranes starting from host cells which express the nucleic acids according to the invention. Such cells preferably express the polypeptides according to the invention in a sufficiently high copy number, so that the polypeptide quantity in a membrane fraction is at least 10 times higher than that in comparable membranes of cells which naturally express acetylcholine receptors; especially preferably, the quantity is at least 100 times higher, very especially preferably at least 1000 times higher.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times higher, especially preferably at least 100 times higher than in a host cell preparation.

The polypeptides according to the invention may also be affinity-purified without fusion component with the aid of antibodies which bind to the polypeptides.

Further subject-matters of the invention are antibodies which specifically bind to the abovementioned polypeptides or receptors. Such antibodies are produced in the customary manner. For example, such antibodies may be produced by injecting a substantially immunocompetent host with such an amount of an acetylcholine receptor polypeptide according to the invention or a fragment thereof which is effective for antibody production, and subsequently obtaining this antibody. Furthermore, an immortalized cell line which produces monoclonal antibodies may be obtained in a manner known per se. If appropriate, the antibodies may be labelled with a detection reagent. Preferred examples of such a detection reagent are enzymes, radiolabelled elements, fluorescent chemicals or biotin. Instead of the complete antibody, fragments may also be employed which have the specific binding properties desired.

The nucleic acid according to the invention can be used in particular for the generation of transgenic invertebrates. These can be employed in test systems which are based on an expression of the receptors according to the invention or variants thereof which deviate from the wild type. This also encompasses all transgenic invertebrates in which expression of the receptors according to the invention or variants thereof changes owing to the modification of other genes or gene control sequences (promoters).

The transgenic invertebrates are generated, for example, in Drosophila melanogaster by P-element-mediated gene transfer (Hay et al. 1997) or in Caenorhabditis elegans by transposon-mediated gene transfer (for example by Tcl, Plasterk 1996).

Subject-matter of the invention are therefore also transgenic invertebrates which contain at least one of the nucleic acids according to the invention, preferably transgenic invertebrates of the species Drosophila melanogaster or Caenorhabditis elegans, and their transgenic progeny. The transgenic invertebrates preferably contain the receptors according to the invention in a form which deviates from the wild type.

The nucleic acid according to the invention can be generated in the customary manner. For example, all of the nucleic acid molecule can be synthesized chemically, or else only short sections of the sequence according to the invention can be synthesized chemically and such oligonucleotides can be radiolabelled or labelled with a fluorescent dye. The labelled oligonucleotides can be used for screening cDNA libraries generated starting from insect mRNA. Clones to which the labelled oligonucleotides hybridize are selected for isolating the DNA in question. After characterization of the DNA which has been isolated, the nucleic acid according to the invention is obtained in a simple manner.

Alternatively, the nucleic acid according to the invention can also be generated by means of PCR methods using chemically synthesized oligonucleotides.

The term “oligonucleotide(s)” as used in the present context denotes DNA molecules composed of 10 to 50 nucleotides, preferably 15 to 30 nucleotides. They are synthesized chemically and can be used as probes.

The nucleic acid according to the invention can be used for isolating and characterizing the regulatory regions which naturally occur in the vicinity of the coding region. Such regulatory regions are thus also subject-matter of the present invention.

The nucleic acid according to the invention allows new active compounds for crop protection or pharmaceutical active compounds for the treatment of humans and/or animals to be identified, such as compounds which alter the conductive properties of the acetylcholine receptors according to the invention as modulators, in particular as agonists or antagonists. To this end, a recombinant DNA molecule comprising the nucleic acid according to the invention is introduced into a suitable host cell. The host cell is grown in the presence of a compound or a sample comprising a variety of compounds under conditions which allow expression of the receptors according to the invention. A change in receptor properties can be detected as described hereinbelow in Example 2. This allows insecticidal substances to be found.

Also, the nucleic acid according to the invention allows compounds to be found which bind to the receptors according to the invention. These too may be employed as insecticides. For example, host cells which contain the nucleic acid according to the invention and express the receptors or polypeptides in question or the gene products themselves are contacted with a compound or a mixture of compounds under conditions which allow the interaction of at least one compound with the host cells, the receptors or the individual polypeptides.

Using host cells or transgenic invertebrates which contain the nucleic acid according to the invention, it is also possible to find substances which alter receptor expression.

The nucleic acid according to the invention, vectors and regulatory regions described hereinabove can also be used for finding genes which encode polypeptides which participate in the synthesis, in insects, of functionally similar acetylcholine receptors. Functionally similar receptors are to be understood as meaning, in accordance with the present invention, receptors which comprise polypeptides which, while differing from the amino acid sequence of the polypeptides described herein, essentially have the same functions.

Information on the Sequence Listing and the Figure

SEQ ID NO: 1 shows the nucleotide sequence of the isolated Db3-cDNA, starting with position 1 and ending with position 1539. SEQ ID NO: 1 and SEQ ID NO: 2 furthermore show the amino acid sequences of the protein derived from the Db3-cDNA sequence.

SEQ ID NO: 3 and SEQ ID NO: 4 show the oligodeoxynucleotides described in Example 1.

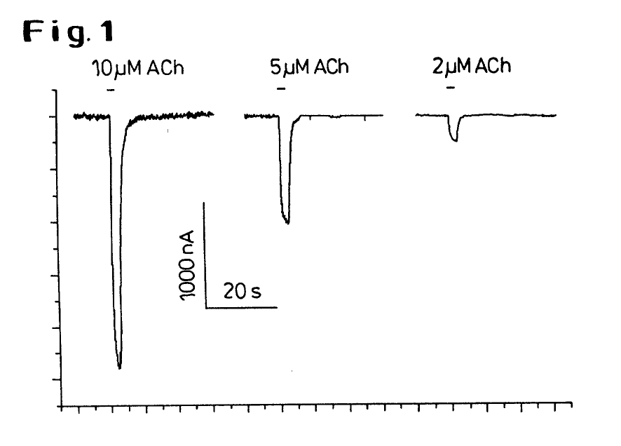

FIG. 1 shows the acetylcholine-induced currents measured on Xenopus oocytes with the aid of whole-cell discharges plotted against time. Currents are shown in nano-ampere, time in seconds. The oocytes had been injected with cDNA expression plasmids which encoded the Drosophila α1, α2 and β3 subunits. The timings of the acetylcholine applications are identified with transverse bars.

### EXAMPLES

### Example 1

Isolation of the Above-described Polynucleotide Sequence

General

Polynucleotides were manipulated by standard methods of recombinant DNA technology (Sambrook et al., 1989). Nucleotide and protein sequences were processed in terms of bioinformatics using the package GCG version 10.0 (GCG Genetics Computer Group, Inc., Madison Wis., USA).

Isolation of Partial Polynucleotide Sequences by Means of PCR

Based on a database search with the protein sequence of the Drosophila melanogaster ARD subunit versus the genomic Drosophila melanogaster database, a nucleic acid region was identified which has 28% identity to ARD at the amino acid level. Oligodeoxynucleotide primers (dg1sense: 5′-TGGCARCCITCICARTAYGA-3′, dg2anti: 5′-CATRATYTTYTCICCICCCAT-3′) were derived on the basis of this partial sequence. RNA was isolated by means of Trizol reagent (Gibco BRL) from Drosophila melanogaster embryos following the manufacturer's instructions. 10 μg of this RNA were employed in a cDNA first-strand synthesis (Superscript preamplification system for cDNA first-strand synthesis, Gibco BRL, following the manufacturer's instructions, reaction temperature 45° C.). Then, in each case 1/100 of the abovementioned first-strand cDNA was employed in a polymerase chain reaction (PCR) with the oligonucleotides dg1sense and dg2anti (Taq DNA polymerase, recombinant, Gibco BRL). The PCR parameters were as follows: 94° C., 1 minute; 35 times (94° C., 30 s; 55° C., 30 s; 72° C., 45 s). This resulted in an approx. 0.6 kb band which was discernible in the agarose gel (1%). The band was subcloned by means of the pCR TOPO kit (Invitrogen).

Isolation of Poly-A-containing RNA from Drosophila melanogaster Tissue, and Construction of the cDNA Libraries.

The RNA for the cDNA library was isolated from Drosophila melanogaster embryos and larvae using Trizol (Gibco BRL) following the manufacturer's instructions. The poly-A-containing RNAs were now isolated from this RNA by purification over Dyna Beads 280 (Dynal). 5 μg of these poly-A-containing RNAs were subsequently employed in the construction of the CDNA library using the λ-ZAP-CMV vector (cDNA Synthesis Kit, ZAP-cDNA Synthesis Kit and ZAP-cDNA Gigapack III Gold Cloning Kit, all from Stratagene).

Isolation of the Complete Polynucleotide Sequences from the cDNA Library

Screening with 10⁶ plaque-forming units was carried out with the aid of the DIG system (all reagents and consumables from Boehringer Mannheim, following instructions in “The DIG System User's Guide for Filter Hybridization”, Boehringer Mannheim). The DNA probe employed was prepared by PCR by means of digoxygenin-labelled dUTP. The hybridizations were performed overnight at 42° C. in DIG Easy Hyb (Boehringer Mannheim). Labelled DNA on nylon membranes was detected by chemoluminescence (CDP-Star, Boehringer Mannheim) using X-ray films (Hyperfilm MP, Amersham). Plaques which had been singled out and which were positive in the hybridization were transferred to plasmids (PCMV) by means of in-vivo excision (Stratagene, ZAP-cDNA Synthesis Kit). For identification, the plasmids isolated were subjected to incipient sequencing by means of T3 and T7 primers (ABI Prism Dye Terminator Cycle Sequencing Kit, ABI, with ABI Prism 310 Genetic Analyzer). The complete polynucleotide sequences of DB3 were determined by primer walking by means of the Cycle Sequencing ABI Prism Dye Terminator Cycle Sequencing Kit, ABI, with ABI Prism 310 Genetic Analyzer.

### Example 2

Expression in Xenopus oocytes of Recombinant Insect Acetylcholine Receptors containing the new Drosophila β3 Subunit.

Oocytes were injected simultaneously with cDNA expression plasmids which encoded the Drosophila α1, β2 and β3 subunits. The a subunits were cloned into pcDNA3, the β3 subunit into pCMV, as described above. After incubation for three to five days, the currents through the oocyte membrane were measured as described using whole-cell discharges (Cooper et al. 1996). To this end, the potential difference over the cell membrane was kept constant at −80 mV and the cells were stimulated with acetylcholine (10 μM). Immediately after the stimulus, strong inward currents were measured, which were typical of the activation of ion channels (FIG. 1). This demonstrates that the new Drosophila β3 subunit forms functional receptors with one of the two coinjected α subunits, or with both.

References

Amar et al. (1995), A nicotinic acetylcholine receptor subunit from insect brain forms a non-desensitizing homo-oligomeric nicotinic acetylcholine receptor when expressed in Xenopus oocytes, Neuroscience Letters 199, 107-110

Bargmann and Kaplan (1998), Signal transduction in the Caenorhabditis elegans nervous system, Ann. Rev. Neurosci., 21, 279-308

Bossy et al. (1988), Conservation of neural nicotinic acetylcholine receptors from Drosophila to vertebrate central nervous systems, EMBO J. 7, 611-618

Breer et al. (1987), Molecular properties and functions of insect acetylcholine receptors, J. Insect Physiol. 33, 771-790

Buckingham et al. (1997), Imidacloprid actions on insect neuronal acetylcholine receptors, J. Exp. Biol. 200, 2685-2692

Changeux et al. (1992), The functional architecture of the nicotinic acetylcholine receptor explored by affinity labelling and site-directed mutagenesis, Quarterly Review of Biophysics 25, 395-432

Claudio et al. (1983), Nucleotide and deduced amino acid sequences of Torpedo californica acetylcholine receptor γ subunit, Proc. Natl. Acad. Sci. USA 80, 1111-1115

Cooper et al. (1996), Eur. J. Pharmacol. 309, 287

Devereux et al. (1984), Nucleic Acids Research 12, 387

Devillers-Thiery et al. (1983), Complete MRNA coding sequence of the acetylcholine binding α-subunit of Torpedo marmorata acetylcholine receptor: a model for the transmembrane organization of the polypeptide chain, Proc. Natl. Acad. Sci. USA 80, 267-2071

Elgoyhen et al. (1997), U.S. Pat. No. 5,683,912

Eastham et al. (1998), Characterisation of a nicotinic acetylcholine receptor from the insect Manduca sexta, Eur. J. Neurosci. 10, 879-889

Hay et al. (1997), P element insertion-dependent gene activation in the Drosophila eye, Proceedings of The National Academy of Sciences of The United States of America 94 (10), 5195-5200

Hermans-Borgmeyer et al. (1986), Primary structure of a developmentally regulated nicotinic acetylcholine receptor protein from Drosophila, EMBO J. 5, 1503-1508

Hermsen et al. (1998), Neuronal nicotinic receptors in the locust Locusta migratoria. Cloning and expression, J. Biol. Chem. 17, 18394-404.

Heinemann et al. (1997), U.S. Pat. No. 5,591,590

Huang et al. (1999), Molecular characterization and imidacloprid selectivity of nicotinic acetylcholine receptor subunits from the peach-potato aphid Myzus persicae, Neurochem., 73, 380-389

Lindstrom et al. (1997), U.S. Pat. No. 5,599,709

Marshall et al. (1990), Sequence and functional expression of a single α subunit of an insect nicotinic acetylcholine receptor, EMBO J. 9, 4391-4398

Matsuda et al. (1998), Effects of the a subunit on imidacloprid sensitivity of recombinant nicotinic acetylcholine receptors, Br. J. Pharmacol. 123, 518-524

Noda et al. (1982), Primary structure of α-subunit precursor of Torpedo californica acetylcholine receptor deduced from cDNA sequence, Nature 299, 793-797

Noda et al. (1983a), Primary structures of β- and δ-subunit precursor of Torpedo californica acetylcholine receptor deduced from cDNA sequences, Nature 301, 251-255

Noda et al. (1983b), Structural homology of Torpedo californica acetylcholine receptor subunits, Nature 302, 528-532

Ortells et al. (1995), Evolutionary history of the ligand-gated ion-channel super-family of receptors, Trends in Neurosience 18, 121-127

Plasterk (1996), The Tc1/mariner transposon family, Transposable Elements/Current Topics in Microbiology and Immunology 204, 125-143

Sambrook et al. (1989), Molecular Cloning, A Laboratory Manual, 2nd ed. Cold Spring Harbor Press

Sawruk et al. (1990a), Heterogeneity of Drosophila nicotinic acetylcholine receptors: SAD, a novel developmentally regulated α-subunit, EMBO J. 9, 2671-2677

Sawruk et al. (1990b), SBD, a novel structural subunit of the Drosophila nicotinic acetylcholine receptor, shares its genomic localization with two α-subunits, FEBS Lett. 273, 177-181

Schloβ et al. (1988), Neuronal acetylcholine receptors of Drosophila: the ARD protein is a component of a high-affinity α-bungarotoxin binding complex, EMBO J 7, 2889-2984

Schoepfer et al. (1990), Brain alpha-bungarotoxin binding protein cDNAs and MAbs reveal subtypes of this branch of the ligand-gated ion channel gene superfamily, Neuron 5, 35-48.

Schulz et al. (1998), Dα3, a new functional α-subunit of nicotinic acetylcholine receptors from Drosophila, J. Neurochem. 71, 853-862

Sgard et al. (1998), Cloning and Functional Characterization of Two Novel Nicotinic Acetylcholine Receptor α subunits from the Insect Pest Myzus persicae; J. Neurochem 71, 903-912

1. Nucleic acid comprising a sequence selected from the group consisting of
(a) the sequence of SEQ ID NO: 1,
(b) subsequences of the sequence defined under (a) which are at least 14 basepairs in length,
(c) sequences which hybridize with the sequence defined under (a),
(d) sequences which have at least 70% identity to the sequence between position 43 and position 1368 of the sequence defined under (a),
(e) sequences which are complementary to the sequence defined under (a), and
(f) sequences which, owing to the degeneracy of the genetic code, encode the same amino acid sequence as do the sequences defined under (a) to (d).

2. Vector comprising at least one nucleic acid according to claim 1.

3. Vector according to claim 2, characterized in that the nucleic acid molecule is operatively linked to regulatory sequences which ensure expression of the nucleic acid in pro- or eukaryotic cells.

4. Host cell comprising a nucleic acid according to claim 1 or a vector according to claim 2 or 3.

5. Host cell according to claim 4, characterized in that it is a pro- or eukaryotic cell.

6. Host cell according to claim 5, characterized in that the prokaryotic cell is E. coli.

7. Host cell according to claim 5, characterized in that the eukaryotic cell is a mammalian or insect cell.

8. Polypeptide encoded by a nucleic acid according to claim 1.

9. Polypeptide which exerts the biological function of an acetylcholine receptor β subunit and which comprises an amino acid sequence having at least 40% identity to the sequence of SEQ ID NO: 2.

10. Acetylcholine receptor comprising at least one polypeptide according to claim 8 or 9.

11. Method of producing a polypeptide according to claim 8 or 9, which comprises
(a) culturing a host cell according to one of claims 4 to 7 under conditions which ensure expression of the nucleic acid according to claim 1, and
(b) obtaining the polypeptide from the cell or the culture medium.

12. Antibody which reacts specifically with the polypeptide according to claim 8 or 9 or with the receptor according to claim 10.

13. Transgenic invertebrate containing a nucleic acid according to claim 1.

14. Transgenic invertebrate according to claim 13, characterized in that it is Drosophila melanogaster or Caenorhabditis elegans.

15. Method of generating a transgenic invertebrate according to claim 13 or 14, wherein a nucleic acid according to claim 1 or a vector according to claim 2 or 3 is introduced.

16. Transgenic progeny of an invertebrate according to claim 13 or 14.

17. Method of generating a nucleic acid according to claim 1, with the following steps:
(a) full chemical synthesis in a manner known per se or
(b) chemical synthesis of oligonucleotides, labelling the oligonucleotides, hybridizing the oligonucleotides with DNA of an insect cDNA library, selecting positive clones and isolating the hybridizing DNA from positive clones or
(c) chemical synthesis of oligonucleotides and amplification of the target DNA by means of PCR.

18. Regulatory region which naturally controls, in insect cells, the transcription of a nucleic acid according to claim 1 and which ensures specific expression.

19. Method of finding new active compounds for crop protection or pharmaceutical active compounds for the treatment of humans or animals, in particular compounds which alter the conductive properties of receptors according to claim 10, with the following steps:
(a) providing a host cell according to any of claims 4 to 7,
(b) culturing the host cell in the presence of a compound or of a mixture of compounds, and
(c) detecting altered conductive properties.

20. Method of finding a compound which binds to receptors according to claim 10, with the following steps:
(a) contacting a host cell according to any of claims 4 to 7, a polypeptide according to claim 8 or 9 or a receptor according to claim 10 with a compound or a mixture of compounds under conditions which allow the interaction of at least one compound with the host cell, the polypeptide or the receptor, and
(b) determining the compound(s) which bind(s) specifically to the receptors.

21. Method of finding compounds which alter the expression of receptors according to claim 10, with the following steps:
(a) contacting a host cell according to any of claims 4 to 7 or a transgenic invertebrate according to claim 13 or 14 with a compound or a mixture of compounds,
(b) determining the receptor concentration, and
(c) determining the compound(s) which specifically affect(s) receptor expression.

22. Use of a nucleic acid according to claim 1, of a vector according to claim 2 or 3, of a host cell according to any of claims 4 to 7, of a polypeptide according to claim 8 or 9, of an acetylcholine receptor according to claim 10, of an antibody according to claim 12, of a transgenic invertebrate according to claim 13 or 14 or of a regulatory region according to claim 18 for finding new active compounds for crop protection or pharmaceutical active compounds for the treatment of humans or animals.


SEQUENCE LISTING (4 sequences)

SEQ ID NO: 1 - DNA, 1539 bp, Drosophila melanogaster
  CDS (43)..(1365)
attcggcacgagggtacatccgaaacaaaggcgcgctgaacaatgacgacgactcccaagataaaggcaccagtttccggtcctggactgccactactgctgcaaatgctaatggggatgcttcttatggggctgacttccgtgccaggcgccactgccaccgcggaccccaagaacgccaatgtcaaggccctggatcgcctccacgccggcctgttcacgaactacgacagcgatgtgcagccggtgttccaaggaacccccacgaacgtgtccctggaaatggtggtcacctacatagacatcgacgagttgaacggcaagctgaccacccactgctggctgaatctccgatggagagacgaggagcgcgtgtggcaaccgtcacaatatgacaacatcacgcagatcactttgaagtccagcgaggtctggaccccccaaatcacactcttcaacggcgacgaaggtggcctgatggccgaaacccaggtgaccctcagccacgatggccacttccggtggatgcctccagccgtgtacacggcctactgcgaactcaacatgctcaactggccccacgacaagcagagctgcaagttgaagatcggctcctggggcctgaaggtcgtcctgccggagaacggcacggcgagaggagagtcccttgaccacgacgacctggttcagtcaccggagtgggaaatcgtggactcgcgagcccactttgtcagtcaggactactacggctacatggagtacactctgacggctcagcggcgctcctccatgtacacggccgtcatctacacacccgcgtcctgcatcgtcatcctggccctctcagccttctggctgcctccccacatgggcggcgagaagatcatgatcaacggcctgctcatcatcgtgatcgccgccttcctcatgtacttcgcccagctcctgccagtgctgtccaacaatactccacttgtggtaatcttctacagcaccagcctgctgtatctgagcgtctccaccatcgtcgaggttctagttctgtacctggccacaggcaagcacaagaggcgcctgccggaggcgctgagaaagctgctgcacgggcacctgggcacgtggctgctgctctcggtgttcagcaccactggcgagtcgcaggcggagaagaccaaagagatggacgagcacccgtacgaggaggcggacgagcaggagtccagtccgctgggcatcaaccacaccgaggtgccgggcgccaaggccaaccagttcgactgggcgctgctggccaccgccgtggaccgcatttccttcgtttccttcagcctggccttcctcattctggccatcaggtgctccgtgtagggatgctcgagactcaaggccacatcccaagccagtgcgcactctgaactagttttgcatttgcgatttcatgtatttaatgtgtgtgcgaacttataattatttaatgatgagacctcgtatggaataaaggacctctgccgaatgtctgcttacaaaaaaaaaaaaaaa

SEQ ID NO: 2 - protein, 441 aa, Drosophila melanogaster
MTTTPKIKAPVSGPGLPLLLQMLMGMLLMGLTSVPGATATADPKNANVKALDRLHAGLFTNYDSDVQPVFQGTPTNVSLEMVVTYIDIDELNGKLTTHCWLNLRWRDEERVWQPSQYDNITQITLKSSEVWTPQITLFNGDEGGLMAETQVTLSHDGHFRWMPPAVYTAYCELNMLNWPHDKQSCKLKIGSWGLKVVLPENGTARGESLDHDDLVQSPEWEIVDSRAHFVSQDYYGYMEYTLTAQRRSSMYTAVIYTPASCIVILALSAFWLPPHMGGEKIMINGLLIIVIAAFLMYFAQLLPVLSNNTPLVVIFYSTSLLYLSVSTIVEVLVLYLATGKHKRRLPEALRKLLHGHLGTWLLLSVFSTTGESQAEKTKEMDEHPYEEADEQESSPLGINHTEVPGAKANQFDWALLATAVDRISFVSFSLAFLILAIRCSV

SEQ ID NO: 3 - DNA, 20 bp, Artificial Sequence
  modified_base 9,12: Primer; n is i at positions 9 and 12
tggcarccntcncartayga

SEQ ID NO: 4 - DNA, 21 bp, Artificial Sequence
  modified_base 13,16: Primer; n is i at positions 13 and 16
catratyttytcnccncccat